(19) United States
(12) Patent Application Publication
(10) Pub. No.: US 2003/0176869 A1
(43) Pub. Date: Sep. 18, 2003

(54) Contact lens removal trauma kit

(76) Inventor: C. Lee Dickerson, Stone Mountain, GA (US)

Correspondence Address: ABBA OPTICAL, INC., C. LEE DICKERSON, PRESIDENT, 2230 CENTRE PARK COURT, STONE MOUNTAIN, GA, 30087, US

(21) Appl. No.: 10/098,584

(22) Filed: Mar. 15, 2002

(51) Int. Cl. A61F 9/00

(52) U.S. Cl. 606/107

(57) ABSTRACT

The Contact Lens Removal Trauma Kit is used to safely remove and store soft or hard contact lenses by a trained individual. The Contact Lens Removal Trauma Kit consists of one dual-purpose removal device for removing soft or hard contact lenses, two amphyls of saline and one contact lens storage case. The Contact Lens Removal Trauma Kit is disposable in nature and is only intended for one single use.

### CROSS-REFERENCE TO RELATED APPLICATIONS

Not Applicable

### STATEMENT REGARDING FEDERALLY SPONSORED RESEARCH OR DEVELOPMENT

Not Applicable

### REFERENCE TO SEQUENCE LISTING, A TABLE, OR A COMPUTER PROGRAM LISTING COMPACT DISK APPENDIX

Not Applicable

### BACKGROUND OF INVENTION

The Contact Lens Removal Trauma Kit was developed to assist persons in the medical field to safely and properly remove, (not insert), soft or hard contact lenses from the eye(s) of a person who is presented unconscious and/or unable to remove contact lenses on their own volition. An immediate need for the medical industry and medical caregivers to have the proper components to safely remove, hydrate and store soft or hard contact lenses was discovered by the inventor, ABBA Optical, Inc.

### BRIEF SUMMARY OF THE INVENTION

The Contact Lens Removal Trauma Kit is used to safely remove and store soft or hard contact lenses by a trained individual in the medical industry. The Contact Lens Removal Trauma Kit consists of one dual purpose removal device for removing soft or hard contact lenses, two amphyls of saline for hydrating the contact lens(es) after removal, and one contact lens storage case for two lenses. The Contact Lens Removal Trauma Kit is disposable and is intended for one use. Because the medical industry and medical caregivers do not have the proper components available to them to safely remove, hydrate and store soft or hard contact lenses, The Contact Lens Removal Trauma Kit was invented to provide all of the necessary elements to allow medical care givers to safely and properly remove soft or hard contact lenses and save them from being discarded so the patient will have aided vision when they are healed or released from medical care.

### BRIEF DESCRIPTION OF THE SEVERAL VIEWS OF THE DRAWING

Not Applicable

### DETAILED DESCRIPTION OF THE INVENTION

The Contact Lens Removal Trauma Kit consists of one disposable, dual-purpose removal device for either soft, or hard contact lenses. One end of the removal device is affixed with a suction cup for removing a hard lens, which is accomplished by holding the removal device perpendicular to the eye and gently placing the suction cup on hard lens, which is resting on the corneal. The hard lens will become adhered to the suction cup and can be lifted from the cornea. The opposite end of the removal is used for removing a soft contact lens. This end has a set of elongated grips, that when placed near the edge of the soft contact lens and squeezed together with the thumb and index finger, will safely grasp the soft lens and allow the remover to lift the lens from the cornea. In addition to the dual-purpose removal device, there are two disposable amphyls of saline to use in hydrating soft or hard lenses after removal, and one storage case for two contact lenses. The components of the kit are made in the following manner: The dual-purpose removal device is created by permanently affixing the hard lens removal suction cup to the end of the soft lens removal device. The Contact Lens Removal Trauma Kit box is assembled and the three components; one dual purpose removal device, two amphyls of saline and one contact lens storage case, is securely placed inside the box. The box is closed and sealed with shrink-wrap.

1. What we claim as our invention is the Contact Lens Removal Trauma Kit.

2. What we claim as our invention is the Contact Lens Removal Trauma Kit and the contents of the kit.
